(19) United States
(12) Patent Application Publication
(10) Pub. No.: US 2025/0360435 A1
(43) Pub. Date: Nov. 27, 2025

(54) AMBIENT-STABLE AFFINITY CHROMATOGRAPHY DEVICE

(71) Applicant: Tecan SP, Inc., Baldwin Park, CA (US)

(72) Inventor: Qi Huang, Camarillo, CA (US)

(21) Appl. No.: 18/669,639

(22) Filed: May 21, 2024

(51) Int. Cl. B01D 15/38; B01D 15/20; B01D 15/22; B01J 20/288

(52) U.S. Cl. CPC B01D 15/3828; B01D 15/20; B01D 15/22; B01J 20/288

(57) ABSTRACT

An affinity chromatography device for capturing biological molecules includes a carrier and a sorbent bed. The sorbent bed has a solid support material and a ligand configured to bind specific biological molecules coupled to the support material. The sorbent bed is immobilized on or in the carrier and is in a dry format. A related method produces an ambient-stable affinity chromatography device for capturing biological molecule. Another related method determines the presence of a specific biological molecule in a sample. The affinity chromatography device can be used in an application in an automated laboratory system for detecting the presence of a specific biological molecule in a sample.

### FIELD OF THE INVENTION

The present invention relates generally to the field of chromatography and the isolation of specific molecules from a mixture based on their affinity for particular ligands or receptors. Specifically, the invention relates to affinity chromatography devices for capturing specific biological molecules via affinity chromatography, which may be used for the purification of monoclonal antibodies (mAb), the isolation of specific subclasses of immunoglobulin G (IgG), and the removal of cross-species IgG contaminants in a biological or medical sample.

### BACKGROUND OF THE INVENTION

Chromatography is a versatile, precise, and widely applicable sample preparation technique focusing on separation and identification based on differences in charge, binding affinities, size, and other characteristics. It is used in analytical chemistry for the pharmaceutical industry, environmental monitoring, forensic science, and for identifying antibodies, proteins, and other biological compounds or to isolate specific species from a sample or clean up a sample before analysis in biological research. The techniques contribute significantly to scientific research and analysis.

Agarose-based resins are typically used in chromatography, especially for affinity chromatography. Affinity chromatography selectively purifies specific molecules based on highly specific ligand-receptor interactions. Beaded agarose is commonly used as the matrix resin for attaching ligands that bind proteins. These ligands are covalently linked to agarose beads, creating an affinity column. Agarose is widely considered the best material for protein purification resins due to its stability, low nonspecific binding, and excellent flow properties in chromatography applications. The natural polysaccharide derived from certain types of red seaweed forms a porous, gel-like matrix that may be positively charged (anion binding) or negatively charged (cation binding). By manipulating buffer conditions (pH and ionic strength), molecules with varying ionic character bind to or dissociate from the solid-phase material allowing selective binding of proteins based on their charge properties. Finally, agarose can tolerate extremes of pH and ionic strength and can withstand high concentrations of denaturants (e.g., urea or guanidine HCl).

The ligands, such as Ni-NTA (nickel-nitrilotriacetic acid), glutathione, streptavidin, Protein A, Protein G, or antibodies, are covalently linked to agarose beads and immobilized thereon. While the sample containing the target protein is passed through the column, the protein of interest selectively binds to the immobilized ligands. After washing away unbound molecules, the protein is then eluted, resulting highly purified protein.

For purifying antibodies, the ligands of choice may be protein A or protein G, which are both bacterial cell wall proteins that have primary binding sites for the Fc region of mammalian immunoglobulin G (IgG) antibodies, including human IgG.

Agarose-based resins in combination with protein A or protein G are known from the prior art and several products using protein A agarose beads are on the market, e.g., a protein A ligand agarose base matrix as an affinity chromatography resin for purification of monoclonal antibody and Fc-fusion proteins by Cytiva.

Antigen-capture substrates other than agarose-based resins in combination with a ligand are rarely used. A few examples can be found in US 2010/0093107 A1 addressing compositions and methods that improve the orientation of antibodies, as well as other Fc-containing proteins and polypeptides, on a surface to enhance interaction between non-Fc portions of the antibodies or other Fc-containing proteins and polypeptides with a sample. It describes an antigen-capture substrate comprising a solid surface coated with a polymer and an antibody-binding protein coupled to the polymer. In various embodiments the polymer is polymethyl methacrylate (PMMA), poly-acrylic acid, poly-styrenesulfonate/poly-2,3-dihydrothieno(3,4-b)-dioxin, polyaniline, poly-styrene-co-methyl methacrylate, polyamide, polyethylene oxide, or polystyrene.

In contrast to affinity chromatography that is primarily used for the purification of specific target molecules from complex mixtures and can be performed on industrial scales, solid-phase extraction (SPE) is usually performed on a laboratory scale, where relatively small volumes of sample are processed, and mainly used for sample preparation, concentrating, and purifying analytes of interest prior to analysis. Affinity chromatography and SPE therefore differ in their application, purpose, and scale. SPE cartridges or disks contain a sorbent material with selective retention properties commonly used in analytical chemistry to extract analytes from biological fluids, environmental samples, or food matrices for subsequent analysis by techniques like chromatography, spectroscopy, or mass spectrometry. Affinity chromatography sorbents are not used for SPE, and manufacturers of affinity chromatography sorbents provide products like Protein A/G and general-purpose affinity chromatography spin columns, His-tag, GST (Glutathione S-transferase), and Strep-tag purification columns, but no devices for SPE.

In SPE the interactions between the sorbent and analytes can be based on a variety of mechanisms including hydrophobic, polar, or ionic interactions. The selectivity is typically more generalized compared to the high specificity of affinity chromatography, allowing for precise purification. While products for SPE often include dry sorbents, all affinity chromatography products are produced, shipped, and stored in an aqueous cooled environment to keep the sorbent in a slurry to maintain the agarose gel particle's structural integrity, and also the heat-sensitive ligands wet and chilled.

### SUMMARY OF THE INVENTION

One limitation of traditional affinity chromatography products, especially agarose-based devices, is their requirement for a wet sorbent bed, which always needs to be immersed in an aqueous buffer due to its material structure. As a consequence, such devices with antigen-capture substrates known in the art require to be tightly sealed in wet packaging and furthermore necessitate refrigeration during packaging, shipping, and storage to keep its functionality for protein purification. Storage is limited even when refrigerated, and shipping is difficult and costly for the following reasons. Shipping with cold packs require insulated containers and gel packs only keep items cool for a limited time, while dry ice may be too cold and is considered hazardous during transportation. The handling of affinity chromatography devices with sorbent slurries in ethanol and water also introduce technical difficulties, e.g., through the oxidation and corrosion of surfaces. Taken together these restrictions make handling traditional affinity chromatography products complicated and expensive and consume a lot of resources.

It is thus an object of the present invention to provide for affinity chromatography devices that can maintain their functionality over time without significant degradation at normal or ambient conditions, such as room temperature and pressure, without the need for special storage conditions like wet packaging and refrigeration. Such ambient-stable affinity chromatography devices shall demonstrate an acceptable ambient shelf-life of months or years and will be used in affinity chromatography.

This is solved by an affinity chromatography device for capturing biological molecules according to claim 1, which comprises a carrier and a sorbent bed comprising a solid support material and a ligand configured to bind specific biological molecules coupled to the support material, wherein the sorbent bed is immobilized on or in the carrier, and wherein the sorbent bed is in a dry format. Further favorable embodiments can, for example, be derived from the respective dependent claims.

The affinity chromatography device comprising a sorbent bed in a dry format has the advantage of allowing its production, shipping and storage to be done at ambient temperatures, whereas its shelf-life can be prolonged to several years without significant losses in binding capacity and performance.

According to an embodiment of the invention, the affinity chromatography device comprises a ligand which is an immunoglobulin-binding protein. The device may comprise a ligand which is an immunoglobulin-binding protein that can bind an Fc region of an immunoglobulin.

According to an embodiment, the device comprises a ligand which is an immunoglobulin-binding protein, whereas the immunoglobulin-binding protein comprises protein A, protein G, protein L, or a truncated protein A, G, or L.

According to an embodiment of the invention, the affinity chromatography device comprises a ligand comprising nickel or nickel-nitrilotriacetic acid (Ni-NTA), glutathione, streptavidin, a protein or antibody or a fragment thereof.

According to the invention the ligand configured to bind specific biological molecules coupled to the support material may be covalently linked and immobilized thereon. According to an embodiment of the invention the ligand coupled to the support material is non-covalently linked and immobilized thereon.

According to an embodiment, the solid support material comprises a polymer suitable to run dry and stay dry without any harm to its integrity or functionality.

According to an embodiment, that polymer is selected from polymethyl methacrylate (PMMA), styrene-divinylbenzene copolymers (PS-DVB), hydroxylated DVB-PS, pyrrolidone-DVB based copolymers, polystyrenesulfonate (PSS), polythiophene dioxin (poly-2,3-dihydrothieno(3,4-b)-dioxin), poly-styrene-co-methyl methacrylate (PS-co-PMMA), polyaniline (PANI), poly(acrylic acid) (PAA), polyethylene (PE), polyvinyl chloride (PVC), polyethylene glycol (PEG), polythiophenes, and polyvinyl alcohol (PVA).

According to an embodiment, the affinity chromatography device comprises a solid support material comprising rigid beads.

According to an embodiment, the solid support material comprises a resin or silica gel suitable to run dry and stay dry without any damage to the integrity or functionality of its structure.

According to an embodiment, the solid support material comprises glass, metal, or ceramics.

According to an embodiment of the invention, the carrier is selected from a plate, a tube, a column, a well, a pipette tip, and a multi-well plate.

According to an embodiment, the carrier may comprise polystyrene or glass.

In an embodiment according to the invention the biological molecules are proteins.

In an embodiment according to the invention the biological molecules are immunoglobulins, including serum or plasma derived IgG, cell cultured monoclonal antibodies (mAb), and Fc fusion proteins, etc.

The object of the present invention is further addressed by a method for producing an ambient-stable affinity chromatography device for capturing biological molecules, comprising coupling a ligand to a solid support material to form a sorbent bed in an aqueous medium, wherein the ligand is configured to bind specific biological molecules, immobilizing the sorbent bed on or in a carrier, and drying the sorbent bed.

According to an embodiment, the method for producing an ambient-stable affinity chromatography device for capturing biological molecules comprises coupling a ligand configured to bind specific biological molecules to a solid support material to form a sorbent bed in an aqueous medium, drying the sorbent bed, and immobilizing the sorbent bed on or in a carrier.

According to an embodiment, the method for producing an affinity chromatography device for capturing biological molecules comprises coupling a ligand configured to bind specific biological molecules to a solid support material to form a sorbent bed in an aqueous medium, drying the sorbent bed to produce a dry powder, reconstituting the dry powder in an aqueous medium, immobilizing the sorbent bed on or in a carrier, and drying the sorbent bed.

According to an embodiment of the invention, drying the sorbent is done in ambient conditions.

The present invention includes a method for determining the presence of a specific biological molecule in a sample, comprising the steps of reconstituting the affinity chromatography device according to the invention in an aqueous medium, contacting the sorbent bed with the sample, wherein the ligand binds specifically to the specific biological molecule when it is present; and determining if the ligand bound the specific biological molecule, wherein if the ligand bound the molecule, the specific biological molecule is determined to be present in the sample.

Finally the present invention includes the use of an affinity chromatography device comprising a carrier and a sorbent bed comprising a solid support material and a ligand coupled to the support material, wherein the sorbent bed is immobilized on or in the carrier, and wherein the sorbent bed is in a dry format, in an automated laboratory system for detecting the presence of a specific biological molecule in a sample, wherein the sorbent bed is reconstituted in an aqueous medium.

Amongst other advantages the present invention will facilitate the production, handling, and use of affinity chromatography devices like columns, plates, tubes, wells, pipette tips, and multi-well plates. Such products may represent highly specialized single-use consumables, engineered to enable efficient affinity chromatography or solid-phase extraction of biological molecules by offering a high specificity and selectivity in capturing and purifying target proteins, even in complex samples. Such molecules may be human antibodies, particularly Immunoglobulin G (IgG), or any Fc-containing proteins from diverse biological and laboratory sample matrices, when the ligand coupled to the stationary phase used is an immunoglobulin-binding protein as e.g., protein A or G.

In practice, these consumables find extensive utility in professional laboratory settings, where they are integrated into workflows utilizing positive pressure extraction instruments. Through a combination of mechanical force and controlled flow dynamics, these instruments facilitate rapid and thorough sample processing, ensuring optimal yield and purity of the target proteins. Subsequently, the purified samples prepared by using the present invention are primed for numerous downstream analytical techniques, including but not limited to ultraviolet (UV) spectroscopy, fast protein liquid chromatography (FPLC), high-performance liquid chromatography (HPLC), or liquid chromatography-mass spectrometry (LC-MS), enabling precise characterization and quantification with exceptional sensitivity and accuracy.

Overall the affinity chromatography device for capturing biological molecules according to the invention will generate better yields regarding both quantity and quality, will simplify affinity chromatography device handling, and allow more processes to be automated, which collectively causes less work and lower operating costs. Furthermore, advantages and conveniences of the invention result from the following description of embodiments.

### BRIEF DESCRIPTION OF THE FIGURES

FIG. 1 is a graph depicting the principle of affinity chromatography for purifying a certain protein using an affinity chromatography sorbent, which in this case is protein A bound to a stationary phase.

FIG. 2 is a graph depicting a narrow bore extraction (NBE) format column with a small sorbent bed size comprising the affinity chromatography sorbent according to the invention.

FIG. 3 shows the standard curve constructed by plotting the absorption values of Table 1 against the known concentrations of IgG.

FIG. 4 is a graph depicting the recovery of IgG shown in Table 3 in % of IgG via extraction with prototype affinity columns with 50 μL of a sorbent comprising PMMA and recombinant protein A. Absorbance measurements at 280 nm are normalized to standard absorption values of human serum IgG (standard curve).

FIG. 5 shows the extraction recovery of IgG in % of 1 mg after prolonged periods of time of dry storing of PMMA-protein A in a narrow bore extraction (NBE) format column. Combined means from three affinity chromatography sorbent lots and error bars indicating combined standard deviations.

FIG. 6 is a diagram of a first method for producing an ambient-stable affinity chromatography device for capturing biological molecules.

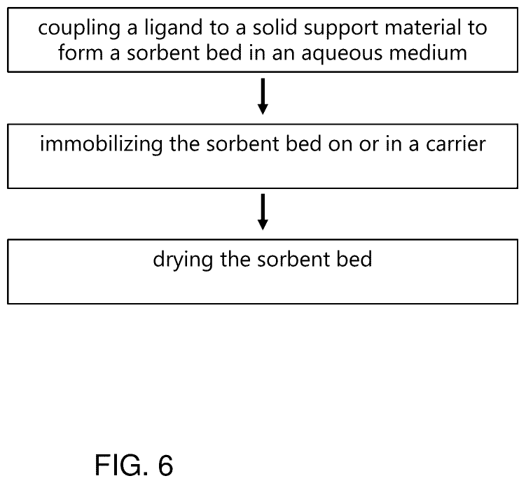

FIG. 7 is a diagram of a second method for producing an ambient-stable affinity chromatography device for capturing biological molecules.

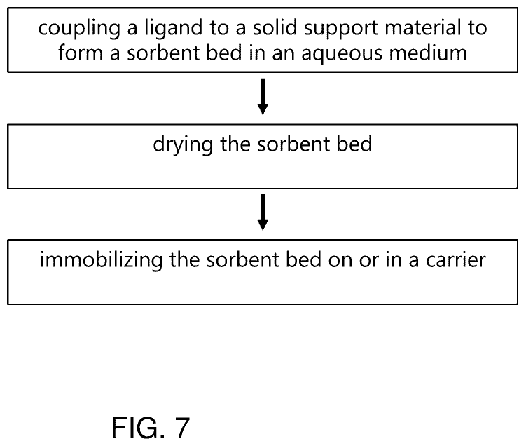

FIG. 8 is a diagram of a third method for producing an ambient-stable affinity chromatography device for capturing biological molecules.

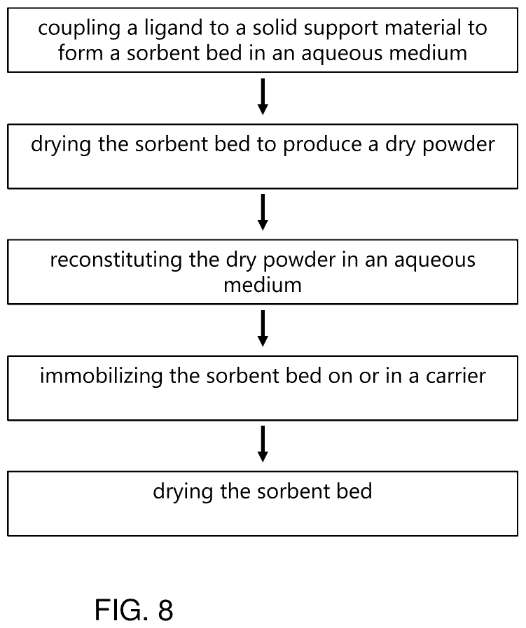

FIG. 9 is a diagram of a method for determining the presence of a specific biological molecule in a sample.

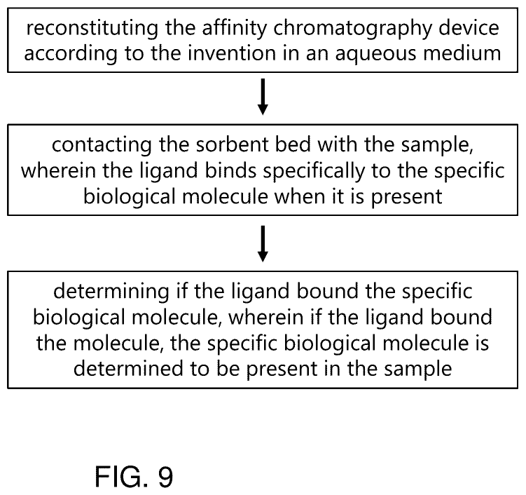

### DETAILED DESCRIPTION

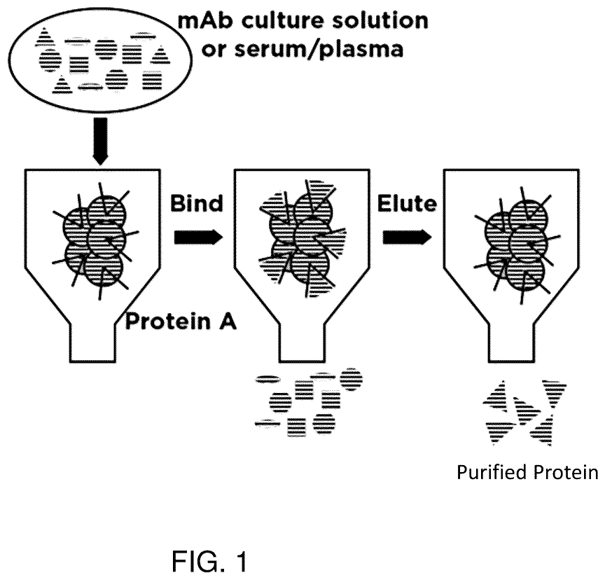

As shown in FIG. 1, an affinity chromatography device comprising a sorbent bed, here as an example comprising protein A as the ligand immobilized on the solid support material, provides a highly specific and efficient means of isolating the desired protein from complex mixtures. Affinity chromatography is a powerful technique utilized in biochemistry and molecular biology for purifying specific proteins from complex mixtures. The principle behind affinity chromatography relies on the selective and reversible binding interactions between the target protein (the molecule to be isolated) and a ligand immobilized on a solid support matrix within a chromatography column or any other carrier.

In the scenario shown, the solid support matrix depicted as round beads together with the ligand depicted as V form the sorbent bed, which can also be referred to as the stationary phase. It is essentially the backbone of the chromatography column, providing a stable platform for the separation process. This sorbent bed is not just any inert material; rather, it is functionalized with a molecule known as protein A. Protein A, in this context, acts as the “bait” molecule. It has a high affinity for the target protein you're interested in purifying. This affinity is typically based on specific molecular interactions such as antigen-antibody binding. When the sample containing the mixture of proteins is applied to the column, the target protein selectively binds to the immobilized protein A ligands while other proteins pass through or bind weakly.

The specific interaction between the target protein and protein A is crucial for the success of the purification process. Once the target protein is captured by the stationary phase, the column is washed to remove any nonspecifically bound contaminants, leaving behind only the target protein bound to protein A. Finally, the purified target protein can be eluted from the column by changing the conditions (such as pH or salt concentration) to disrupt the binding between the protein A and the target protein, thereby releasing the purified protein in its native form.

Instead of using protein A, the ligand used may be protein G or L, nickel, streptavidin, or any other suitable molecule or parts thereof.

Nickel bond sorbent has the affinity to bioengineered proteins with poly-histidine tags. The poly-his tags are strategically placed as a handle just for purification purposes.

Streptavidin has a natural affinity toward biotin. Molecules with a biotin tag may be extracted out by the streptavidin bond sorbent.

According to an embodiment of the invention, the device may comprise a ligand binding the biological molecules of interest through ion exchange, i.e., strong cation exchange (SCX), weak cation exchange (WCX), strong anion exchange (SAX), or weak anion exchange (WAX).

In an embodiment of the invention, the device comprises a ligand binding the biological molecules of interest through reverse phase chromatography making use of nonpolar stationary phases and polar mobile phases to separate molecules based on their hydrophobicity. Examples of materials used as stationary phases in reverse phase chromatography are Atlas (hydrophilic-lipophilic balance, HLB), polystyrene-divinylbenzene (PS-DVB), and C18 (coated with octadecyl chains), each with its own characteristics and suitability for different applications.
E1. An affinity chromatography device for capturing biological molecules comprising
a carrier, and
a sorbent bed comprising
a solid support material; and
a ligand configured to bind specific biological molecules coupled to the support material,
wherein the sorbent bed is immobilized on or in the carrier, and
wherein the sorbent bed is in a dry format.
E2. The affinity chromatography device of E1, wherein the ligand coupled to the solid support material comprises nickel or nickel-nitrilotriacetic acid (Ni-NTA), glutathione, streptavidin, a protein or antibody or a fragment thereof.
E3. The affinity chromatography device of E1 or E2, wherein the ligand coupled to the solid support material is an immunoglobulin-binding protein.
E4. The affinity chromatography device of E3, wherein the immunoglobulin-binding protein can bind an Fc region of an immunoglobulin.
E5. The affinity chromatography device of E3 or E4, wherein the immunoglobulin-binding protein comprises protein A, protein G, protein L, or a truncated protein A, protein G, or protein L.
E6. The affinity chromatography device of E3 or E4, wherein the immunoglobulin-binding protein is protein A.
E7. The affinity chromatography device of any of E1 to E6, wherein the ligand coupled to the solid support material is covalently or non-covalently linked and immobilized thereon.
E8. The affinity chromatography device of any of E1 to E7, wherein the solid support material comprises a polymer.
E9. The affinity chromatography device of E8, wherein the polymer is selected from polymethyl methacrylate (PMMA), styrene-divinylbenzene copolymers (PS-DVB), hydroxylated DVB-PS, pyrrolidone-DVB based copolymers, polystyrenesulfonate (PSS), polythiophene dioxin (poly-2,3-dihydrothieno(3,4-b)-dioxin), poly-styrene-co-methyl methacrylate (PS-co-PMMA), polyaniline (PANI), poly(acrylic acid) (PAA), polyethylene (PE), polyvinyl chloride (PVC), polyethylene glycol (PEG), polythiophenes, and polyvinyl alcohol (PVA).
E10. The affinity chromatography device of E8, wherein the polymer is polymethyl methacrylate (PMMA).
E11. The affinity chromatography device of any of E1 to E10, wherein the solid support material comprises rigid beads.
E12. The affinity chromatography device of any of E1 to E11, wherein the carrier is selected from a plate, a tube, a column, a well, a pipette tip, and a multi-well plate.
E13. The affinity chromatography device of any of E1 to E12, wherein the carrier comprises polystyrene or glass.
E14. The affinity chromatography device of any of E1 to E13, wherein the biological molecules are proteins.
E15. The affinity chromatography device of any of E1 to E14, wherein the biological molecules are immunoglobulins.
E16. A method for producing an affinity chromatography device for capturing biological molecules,
the method comprising:
coupling a ligand to a solid support material to form a sorbent bed in an aqueous medium, wherein the ligand is configured to bind specific biological molecules;
immobilizing the sorbent bed on or in a carrier; and
drying the sorbent bed.
E17. A method for producing an affinity chromatography device for capturing biological molecules,
the method comprising:
(a) coupling a ligand to a solid support material to form a sorbent bed in an aqueous medium, wherein the ligand is configured to bind specific biological molecules;
(b) drying the sorbent bed to produce a dry powder;
(c) reconstituting the dry powder in an aqueous medium;
(d) immobilizing the sorbent bed on or in a carrier; and
(e) drying the sorbent bed.
E18. The method for producing an affinity chromatography device of any of E16 to E17, wherein the drying the sorbent is in ambient conditions.
E19. A method for determining the presence of a specific biological molecule in a sample, the method comprising:
(a) reconstituting the affinity chromatography device of any of E1 to E15 in an aqueous medium;
(b) contacting the sorbent bed with the sample, wherein the ligand binds specifically to the specific biological molecule when it is present; and
(c) determining if the ligand bound the specific biological molecule, wherein if the ligand bound the molecule, the specific biological molecule is determined to be present in the sample.
E20. Use of an affinity chromatography device according to any of E1 to E15 in an application in an automated laboratory system for detecting the presence of a specific biological molecule in a sample, wherein the sorbent bed is reconstituted in an aqueous medium.
E21. An automated laboratory system for detecting the presence of a specific biological molecule in a sample comprising the affinity chromatography device according to any of E1 to E15, wherein the sorbent bed is reconstituted in an aqueous medium.

### Definitions

In the present invention, the terms below are defined as follows:

“Affinity chromatography sorbent” or “sorbent bed” are specifically used for affinity chromatography applications, where the stationary phase contains ligands that have high affinity and specificity for the target molecule(s). Affinity chromatography sorbents are primarily used for the purification of specific target molecules from complex mixtures, whereas the target protein binds specifically to the immobilized ligand while non-specific proteins pass through. An “affinity chromatography sorbent” or “sorbent bed” is a structured arrangement of material within a chromatography device designed to serve as the stationary phase in chromatographic processes to selectively retain specific substances from a fluid sample passing through it, facilitating the separation, purification, or analysis of target compounds based on their differential interactions with the sorbent material.

“Dry” and “dryness” relates to the physical condition characterized by a deficiency or absence of moisture or water content, or any other liquid content in a substance, environment, or system. In scientific terms, dryness is often quantified by measuring the relative humidity, which represents the amount of water vapor present in the air relative to the maximum amount the air can hold at a given temperature. Ranges of dryness can vary depending on the context, but generally, environments with relative humidity levels below 30% are considered dry. Extremely dry conditions may register even lower relative humidity levels, sometimes dropping below 10%.

“Solid support material” or “support material” refers to the matrix within the affinity chromatography column or any other carrier, e.g., plate, tube, column, well, pipette tip, or multi-well plate. This phase is specifically designed to have immobilized ligands or receptors that selectively interact with the target molecules or analytes of interest in the sample being separated. The support material plays a crucial role in separating the target molecules from the rest of the sample based on their affinity for the immobilized ligands, allowing for purification or analysis of the target molecules.

“Beads” refers to small, spherical or near-spherical objects typically composed of a variety of materials such as polymers, resins, silica gel, glass, metals, or ceramics.

Beads can range in size from a few micrometers to several millimeters in diameter. The spherical shape of beads facilitates uniform packing and distribution, making them useful for tasks like column chromatography or as support matrices for immobilized enzymes or antibodies.

A “ligand” as used in molecular biology typically relates to a molecule that binds specifically to a biomolecule such as a protein or nucleic acid, whereas it may alter its structure or activity. Ligands may act as signaling molecules, modulators of enzyme activity, or regulators of gene expression. They can include hormones, neurotransmitters, substrates, inhibitors, activators, and cofactors. In receptor-ligand interactions, ligands bind to cell surface receptors, where they may trigger a cellular response. In enzyme-substrate interactions, ligands bind to enzymes and may initiate biochemical reactions. More generally, a ligand is a molecule or ion that binds specifically to a central atom, typically a metal ion, forming a complex. The binding of the ligand to the central atom is usually through coordination bonds, where the ligand donates one or more pairs of electrons to the central atom. This interaction can result in the formation of stable coordination complexes with distinct chemical and physical properties.

“Biological molecules”, also known as “biomolecules”, refers to a diverse group of molecules that are essential for life processes in living organisms, being fundamental to the structure, function, and regulation of cells and organisms. These molecules are primarily composed of carbon, hydrogen, oxygen, nitrogen, and other elements such as phosphorus and sulfur. Biological molecules encompass a wide range of classes, including carbohydrates, lipids, proteins, nucleic acids, and smaller organic molecules such as vitamins and coenzymes. Each class of biological molecule serves specific functions in living organisms. Carbohydrates, for instance, provide energy and serve as structural components. Lipids are crucial for cell membrane structure, energy storage, and signaling. Proteins play roles in enzymatic catalysis, structural support, transportation, and regulation of cellular processes. Nucleic acids, including DNA and RNA, carry genetic information and are involved in protein synthesis and gene regulation.

“Immunoglobulins” (abbreviated to Ig), also known as antibodies, are glycoprotein molecules produced by B lymphocytes (B cells) of the immune system in response to the presence of foreign substances known as antigens. Structurally, immunoglobulins are Y-shaped molecules composed of four polypeptide chains: two identical heavy chains and two identical light chains, held together by disulfide bonds. The amino acid sequences of the variable regions of these chains confer antigen-binding specificity to each antibody. The five main classes of immunoglobulins in humans are IgM, IgG, IgA, IgD, and IgE, each with distinct structural and functional properties that contribute to the diverse and coordinated adaptive immune response, recognizing and binding to specific antigens, thereby marking them for destruction or neutralization by other components of the immune system.

“Antibodies” (abbreviated to Ab) relate to gamma globulin proteins found in the blood or other bodily fluids of vertebrates and can activate various effector mechanisms, including opsonization, complement activation, and antibody-dependent cellular cytotoxicity (ADCC), to eliminate pathogens, such as bacteria and viruses, or infected cells. The term “antibody” as used here comprises monoclonal antibodies, polyclonal antibodies, and multispecific antibodies, e.g. bispecific antibodies. The term “antibody” is used interchangeably with “immunoglobulin”.

A “protein” refers to a complex macromolecule composed of one or more chains of amino acids linked together by peptide bonds. These chains fold into specific three-dimensional structures determined by the sequence of amino acids, as well as interactions with other molecules and the surrounding environment. Proteins are essential biomolecules that play diverse roles in living organisms, including serving as structural components, enzymes catalyzing biochemical reactions, transporters of molecules within cells and across membranes, receptors for signaling molecules, and regulators of gene expression and cell function.

A “truncated protein” refers to a protein molecule that is shorter than its full-length, native form due to a deletion or removal of one or more amino acid residues from its sequence. This deletion can occur during protein synthesis, post-translational modifications, or as a result of mutations in the gene encoding the protein. Truncated proteins may lack functional domains or motifs present in the full-length protein, leading to alterations in their biological activity or interaction with other molecules.

“Covalently linked” relates to the scientific term where two molecules or atoms are bonded together by sharing pairs of electrons between their outer electron shells.

Covalent bonds are characterized by the sharing of electrons to achieve a stable electron configuration, typically resulting in the formation of a strong and stable bond. In the context of molecules as used here, covalently linked molecules share one or more pairs of electrons to form a covalent bond between them. This type of bond is often found in organic molecules, where atoms of carbon, hydrogen, oxygen, nitrogen, and other elements form covalent bonds to create diverse structures, ranging from simple molecules like hydrogen gas to complex polymers such as proteins, DNA, and organic compounds.

A “carrier” refers to an entity that transports, delivers, facilitates the movement, or holds another substance. Carriers may undergo conformational changes or specific interactions with the transported substance, thereby serving various functions, including facilitating the solubility or stability of substances, enhancing their delivery, or aiding in separation and purification processes, however in the context here carriers may hold together and facilitate the transfer of matter across different systems without undergoing significant alteration itself. Carriers can take various forms, including vessels such as e.g., a plate, a tube, a column, a well, a pipette tip, and a multi-well plate.

“Reconstituting” relates to the process of restoring a substance for use to its original or intended state, ensuring its stability, solubility, and functionality is maintained according to desired specifications, by adding a solvent or reagent in the form of an aqueous medium to a dried or concentrated form. This process is commonly employed in various fields such as pharmaceuticals, biotechnology, and food science. For example, in molecular biology, reconstituting a lyophilized (freeze-dried) enzyme or reagent involves adding a specific buffer or solvent to restore its activity for use in experiments.

The term “aqueous medium” refers to a solution or environment in which water serves as the solvent or typically where water constitutes the primary medium or solvent in which chemical reactions, biological processes, or physical interactions occur. Water plays a significant role in facilitating various processes or interactions in buffer solutions and reagent solutions, e.g., in a solvent with 20% ethanol in water.

### Examples

The present invention will be understood more clearly on reading the following examples, which illustrate the invention non-restrictively:

As an embodiment according to the invention, a protein A sorbent bed with its solid support material polymethyl methacrylate (PMMA) was prepared as a slurry in 20% ethanol in water. This slurry was then dried at ambient temperature and pressure (ambient conditions) to obtain a dry powder format. The resulting affinity chromatography sorbent has shown to be suitable to stay dry, be reconstituted and run dry one or several times without damaging the integrity of the polymer and ligand structure. This dry powder solved some of the difficulties common during the production of affinity chromatography devices. E.g., while the handling of wet sorbent slurries in ethanol and water triggered oxidation and corrosion of surfaces, the dried affinity chromatography sorbent eliminated these restrictions and allowed the production and handling of affinity chromatography devices to become simpler and to use fewer resources.

In addition, stability studies of a narrow bore extraction (NBE) format column by Tecan of the affinity chromatography sorbent comprising PMMA and protein A in powder demonstrated a greater than 3-year shelf-life under ambient conditions without compromising functionality, including specificity and yield. Such a product according to the invention therefore allows packaging, shipping, and storage without refrigeration and without a sealed wet packaging.

It may be used for the purification and identification of antibody and other proteins e.g., by mAb research laboratories in the biopharma industry, or academic, diagnostic and clinical labs. For automation utility in the biopharma industry, it may for example be coupled with the automated 96-well plate processor Resolvex® A100 or A200 by Tecan, making it suitable for high-throughput workflows for submilligram to milligram antibody capture and purification.

### Ambient-Stable Column and Resolvex® Positive Pressure Instruments

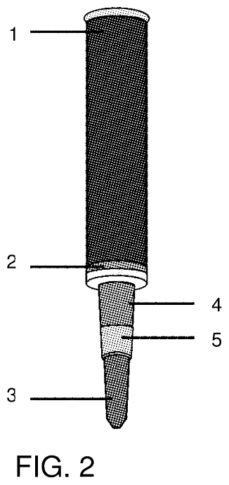

FIG. 2 depicts a narrow bore extraction (NBE) format column by Tecan with a small sorbent bed size comprising the affinity chromatography sorbent according to the invention. The Tecan NBE format column is a specialized column used in chromatography techniques, particularly in liquid chromatography applications.

The chromatography column boasts several key features designed to enhance its functionality and performance. At its core lies the reaction chamber 1, a specialized compartment that facilitates in-column reactions, enabling efficient sample processing and analysis without the need for additional equipment.

To ensure optimal performance, the column is equipped with a built-in filter 2, strategically positioned to facilitate initial matrix cleanup. This crucial component prevents affinity chromatography sorbent overload, thereby enhancing the purity and integrity of the separated samples.

The column is designed with a narrow bore 3, meaning it has a smaller diameter compared to traditional chromatography columns. This narrow bore outlet is strategically designed to direct eluted volumes with precision. By channeling small elution volumes, this feature enhances analyte concentration, thereby facilitating downstream analysis and detection. This is particularly useful when dealing with limited sample volumes or when high sensitivity is required. Despite its smaller size, the NBE format column maintains high resolution and efficiency in separating components of a mixture. This is achieved through careful design and packing of the column material, ensuring optimal flow and interaction between the sample components and the stationary phase.

Central to its efficiency is the utilization of microparticulate sorbent 5, a high-performance material that enables the construction of columns with small bed sizes without compromising on flow rates. This feature allows for rapid and efficient separation of analytes while maximizing sample throughput. This not only saves on solvent costs but also contributes to environmental sustainability by reducing waste.

To further optimize sample handling and retention, the column features an air lock 4. This innovative design prevents sample breakthrough until positive pressure is applied, ensuring precise control over the chromatographic process and minimizing the risk of sample loss or contamination.

The NBE format column by Tecan with a small sorbent bed size is well suited to be filled with the affinity chromatography sorbent according to the invention, allowing its assembly, shipping and storage to be done at ambient temperatures, prolonging its shelf-life to several years without significant losses in binding capacity and performance.

The NBE format column is designed for high-throughput sample preparation in laboratories and typically has a 96-well plate format, which aligns with standard microplate dimensions. It consists of individual wells arranged in an 8×12 grid, allowing simultaneous processing of multiple samples. The column design ensures compatibility with automated liquid handling systems, such as those offered by Tecan. This allows for streamlined and reproducible sample preparation and analysis workflows, enhancing efficiency and throughput in chromatography experiments. As an example, an ambient-stable affinity chromatography sorbent according to the invention may be applied in the NBE format column or any other consumable to be used with the benchtop positive pressure instrument Resolvex® M10 and the automated 96-well plate processor Resolvex® A100 or A200 by Tecan, making it suitable for high-throughput workflows.

The NBE format column by Tecan may have volumes of 1 mL, 3 mL, or 6 mL, whereas the ligands may be protein A or G, or Nickel bond. The affinity chromatography sorbent fill volumes range from 50 μL to 1000 μL, with a binding capacity from 1 mg to 20 mg.

The NBE format column is commonly used in various applications, including sample purification, solid-phase extraction (SPE), drug metabolism studies, and protein purification, with applications in various fields such as pharmaceuticals, biotechnology, and research laboratories where precise and efficient separation of components in a sample is required. It is particularly valuable in high-throughput screening assays and analytical methods requiring rapid analysis of numerous samples.

Sample Preparation Protocol with Protein A Affinity Columns in NBE Format

This protocol outlines the steps for using the ambient-stable affinity chromatography sorbents to separate and purify target molecules from a sample. It involves buffer exchange, sample loading, washing, elution, and analysis steps to achieve the desired purification and isolation of the target molecules.
1. Buffer Exchange:
   Rinse the column with 300 μL of 20% ethanol in H2O.
   Rinse the column with 300 μL of deionized (DI) H2O.
   Rinse the column with 300 μL of loading buffer.
2. Sample Loading:
   Load samples in 300 μL of pH 7.1 PBS buffer (66.7 mM).
   Collect the filtrate.
3. Sample Reload:
   Reload the collected filtrate onto the column two times.
4. Washing:
   Wash the column with 300 μL of loading buffer.
   Wash the column with 300 μL of DI H2O.
5. Elution:
   Elute the column with two times 300 μL of pH 3.3 acetate buffer (20 mM).
6. Analysis:
   Collect each round of elution.
   Analyze each collected fraction for absorption at 280 nm.

### Establishing Analytical Method for IgG Quantification

To effectively advance research and development objectives, and to assess feasibility and the practicality of the invention, several key lab testing goals have been established:

Firstly, a quantifiable IgG analytical method was established within a relevant concentration range. This method served as a crucial tool for accurately measuring IgG levels, providing essential data for research assessments.

In tandem with this, the feasibility of manually dispensing the affinity chromatography sorbent into the Tecan narrow bore extraction (NBE) format columns was assessed. The NBE format column is designed for high-throughput sample preparation in laboratories and typically has a 96-well plate format, which aligns with standard microplate dimensions. It consists of individual wells arranged in an 8×12 grid, allowing simultaneous processing of multiple samples. The column design ensures compatibility with automated liquid handling systems, making it suitable for high-throughput workflows. The Tecan NBE format column is commonly used in various applications, including sample purification, solid-phase extraction (SPE), drug metabolism studies, and protein purification.

Understanding the practicality of this process is essential for streamlining workflows and optimizing efficiency in subsequent stages.

Moreover, IgG recovery using prototype columns and the automation feasibility of the workflow were evaluated. This step was pivotal in gauging the scalability and potential automation of processes, laying the groundwork for future advancements.

Lastly and most importantly, the residual efficacy of the affinity chromatography sorbent after storage under different conditions was evaluated. By comparing stability at ambient temperature versus refrigeration over a defined period of time, insights into shelf life and storage requirements were sought. This assessment is integral to ensuring product integrity during storage and transportation, thereby informing optimal handling protocols.

In essence, these lab testing goals were strategically designed to address critical aspects of the R&D assessment, ranging from method establishment and process optimization to product stability and quality assurance. Through this comprehensive testing and analysis, the current invention has progressed, and the efficiency and reliability of processes were significantly enhanced. The lab testing goals were achieved as follows:

The establishment of a quantifiable IgG analytical method within a relevant concentration range was accomplished. A linear range of 0.03-2.0 mg/mL was established using Spark® by Tecan, ensuring accurate measurement and analysis of IgG levels.

The feasibility of dispensing the sorbent into the NBE columns manually was assessed. It was confirmed through meticulous experimentation that NBE columns could be efficiently filled with 50 μL of sorbent slurry, maximizing the column's capacity while ensuring uniformity and precision in the process.

The IgG recovery with the prototype columns was evaluated, focusing on developing a workflow feasible for automation. Remarkably, a recovery rate of over 90% was achieved with an automation-friendly workflow, paving the way for streamlined and efficient processes in future applications.

The residual efficacy of the affinity chromatography sorbent after storage at ambient conditions compared to 4° C. for six days was investigated. Encouragingly, all samples retained nearly all efficacy in terms of IgG recovery, demonstrating the stability and reliability of the affinity chromatography sorbent even under less-than-ideal storage conditions.

### Measuring the Concentration of Human Serum IgG

A standard curve using absorption at 280 nm was created using the Spark® multimode microplate reader platform by Tecan to facilitate the accurate spectrophotometric measurement and precise quantification of human serum IgG concentration.

A series of standard solutions containing known concentrations of human serum IgG was prepared. These solutions served as reference points for the standard curve.

Using the Spark® multimode microplate reader platform set to a wavelength of 280 nm, the absorption spectra of the standard solutions were measured. This wavelength is chosen as it corresponds to the absorption peak of proteins, including IgG.

TABLE 1
Data from two absorption measurements at 280 nm (samples
1 and 2) used to plot the standard curve of FIG. 1 and calculating
concentrations of IgG. STD; standard deviation.

| | sample 1 | sample 2 | | | |
|---|---|---|---|---|---|
| Concentration | absorption | absorption | | | |
| mg/mL | at 280 nm | at 280 nm | Mean | Net/Standard | STD |
| 0 | 0.056 | 0.054 | 0.055 | | |
| 0.032 | 0.086 | 0.084 | 0.085 | 0.030 | 0.002 |
| 0.063 | 0.109 | 0.110 | 0.110 | 0.055 | 0.001 |
| 0.125 | 0.163 | 0.170 | 0.166 | 0.111 | 0.005 |
| 0.25 | 0.298 | 0.293 | 0.295 | 0.241 | 0.004 |
| 0.5 | 0.547 | 0.554 | 0.550 | 0.495 | 0.005 |
| 1 | 1.027 | 1.030 | 1.028 | 0.973 | 0.002 |
| 2 | 1.960 | 1.944 | 1.952 | 1.897 | 0.011 |

The absorption spectra of the unknown human serum samples were then measured using the same spectrophotometer settings (Spark® by Tecan). The absorption values obtained were used to interpolate the corresponding concentrations of IgG from the standard curve.

Finally, the concentrations of IgG in the unknown samples were calculated based on the interpolation from the standard curve.

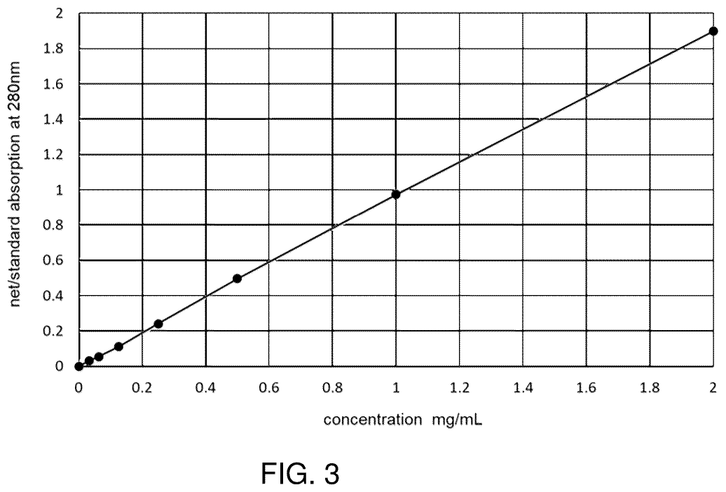

FIG. 3 shows the standard curve constructed by plotting the absorption values of Table 1 against the known concentrations of IgG.

Assess IgG Recovery with Prototype Columns with a Workflow Suitable for Automation

Prototype Tecan narrow bore extraction (NBE) format columns with 50 μL sorbent comprising PMMA and recombinant protein A were used to measure the recovery of IgG via extraction. The spherical, monodispersed particles of PMMA have a size of about 85 μm. Protein A is coupled through epoxy activation and shows a dynamic binding capacity of over 35 mg hlgG/mL resin. The sorbent is stored at 2 to 8° C. in 20% ethanol. It is stable in aqueous buffers commonly used in protein A chromatography; 100-500 mM NaOH, 0.1 M 20% sodium citrate/HCl (pH 3), 6 M Gua-HCl, 8 M urea, 20% ethanol, 2% benzyl alcohol.

The recovery consistency of the 50 μL protein A in NBE format columns was demonstrated by extracting various amounts of IgG from a 300 μL IgG standard solution. The “extracted sum” in Table 2 shows the summation of two measurements of the elution of two 300 μL IgG standard solutions at each concentration of the calibration curve.

TABLE 2
Data for the generation of a standard curve of human serum
IgG after extraction with prototype affinity columns with
50 μL sorbent comprising PMMA and recombinant protein
A, demonstrating recovery consistency of the 50 μL protein
A in NBE format columns when extracting various amounts of IgG.

| Concentration | Standard absorption | Extracted | Recovery |
|---|---|---|---|
| mg/mL | at 280 nm | Sum | in % |
| 0 | 0 | 0 | |
| 0.032 | 0.030 | 0.024 | 81.0 |
| 0.063 | 0.055 | 0.058 | 105.6 |
| 0.125 | 0.111 | 0.105 | 94.0 |
| 0.25 | 0.241 | 0.225 | 93.4 |
| 0.5 | 0.495 | 0.454 | 91.6 |
| 1 | 0.973 | 0.897 | 92.2 |
| 2 | 1.897 | 1.732 | 91.3 |

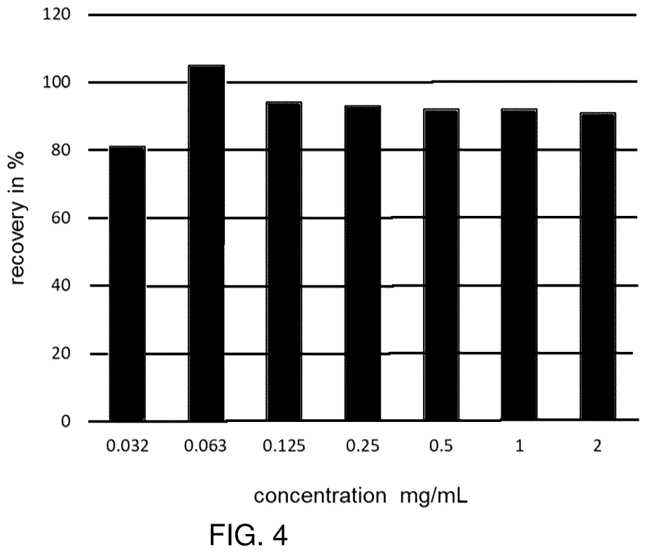

FIG. 4 is a graph depicting the recovery of IgG shown in Table 3 in % of IgG via extraction with prototype affinity columns with 50 μL sorbent comprising PMMA and recombinant protein A. Absorbance measurements at 280 nm are normalized to standard absorption values of human serum IgG (standard curve).

Stress Test for Sorbent Bed in Prototype Affinity Columns with 50 μL Sorbent Comprising PMMA and Recombinant Protein A
Column type 1: Columns filled with 50 μL affinity chromatography sorbent comprising PMMA and recombinant protein A, covered loosely with aluminum foil, placed on the lab bench at room temperature for 6 days.
Column type 2: Columns filled with 50 μL affinity chromatography sorbent comprising PMMA and recombinant protein A, covered loosely with aluminum foil, placed in a refrigerator at 4° C. for 6 days.
Column type 3: Previously used columns rinsed with 600 μL pH 3.3 20 mM acetate buffer and then 600 μL phosphate buffer (pH 7.1 66.7 mM), covered loosely with aluminum foil, placed in a refrigerator at 4° C. for 6 days.

IgG recovery was evaluated on a loading of 300 μL of 1 mg/mL IgG in phosphate buffer (pH 7.1 66.7 mM).

TABLE 3
Recovery in % of IgG in stress test for sorbent bed in
prototype affinity columns with 50 μL affinity chromatography
sorbent comprising PMMA and recombinant protein A

| | Column type | | |
|---|---|---|---|
| | 1 | 2 | 3 |
| | 89.2 | 91.7 | 85.7 |
| | 90.0 | 89.7 | 89.8 |
| | 92.5 | 94.6 | 89.0 |
| | 92.6 | 92.1 | 91.0 |
| | | | 80.0 |
| | | | 90.8 |
| | | | 93.9 |
| | | | 91.8 |
| mean % | 91 | 92 | 89 |
| STD | 1.7 | 2.0 | 4.3 |

TABLE 4
Recovery of IgG via extraction with prototype affinity columns with 50 μL
of a sorbent comprising PMMA and recombinant protein A. Absorbance measurements
at 280 nm (samples 1, 2, and 3) normalized to standard absorption values of
human serum IgG (standard curve) used for calculating recovery in % of IgG.

| Conc. | Standard | sample | sample | sample | | | Recovery |
|---|---|---|---|---|---|---|---|
| mg/mL | UV Abs | 1 | 2 | 3 | Mean | STD | % |
| 0 | 0 | 0 | 0 | 0 | 0 | | |
| 0.032 | 0.03 | 0.03 | 0.02 | 0.02 | 0.02 | 0.001 | 81 |
| 0.063 | 0.06 | 0.05 | 0.06 | 0.06 | 0.06 | 0.006 | 105 |
| 0.125 | 0.11 | 0.09 | 0.11 | 0.12 | 0.11 | 0.011 | 94 |
| 0.25 | 0.24 | 0.22 | 0.22 | 0.23 | 0.23 | 0.004 | 93 |
| 0.5 | 0.50 | 0.46 | 0.45 | 0.45 | 0.45 | 0.004 | 92 |
| 1 | 0.97 | 0.91 | 0.88 | 0.90 | 0.90 | 0.013 | 92 |
| 2 | 1.90 | 1.72 | 1.75 | 1.72 | 1.73 | 0.017 | 91 |

Assess Residual Efficacy of the Sorbent after Storage at Ambient Condition

The results of the stability assessment are indicative to product shelf life and shelf condition, thus relevant to storage conditions, and transportation requirements.

Three lots of NBE columns with 50 μL affinity chromatography sorbent comprising PMMA and recombinant protein A were produced from three different lots of sorbents. The goal of the study is to verify the functional stability of these columns over a set period of time when the columns under the test were stored in an oven at 37° C. A set of 12 columns from each lot were taken out of the oven at the set test day. Functional tests were performed via human serum IgG binding recovery with 1 mg per column loading. The study design is as following:

### Testing Duration and Functional Testing Schedule:

1. For accelerated aging (AA), per Q10 rule calculation, using template 300-04-DEV-T10, an aging factor of 4 was used according to known product criticality data. NBE columns with 50 μL affinity chromatography sorbent comprising PMMA and recombinant protein A were aged, and testing was carried out for 15 weeks. This represents 24 months of real time aging under ambient temperature as shown in Table 5.

$$Accelerated Aging Time = \frac{Desired Shelf Life}{{Q}_{10}^{\frac{{r}_{AA} - {T}_{S}}{10}}}$$

TABLE 5
Q10 accelerated aging calculation

| | | Unit of |
|---|---|---|
| Input Parameter | Value | Measure |
| Desired Shelf Life | 24 | month |
| Accelerated Aging Temperature, TAA | 37 | Celsius |
| (Storage Temperature) Ambient | 23 | Celsius |
| Temperature, Ts | | |
| Aging Factor, Q10 | 4 | Criticality |

| | | Unit of |
|---|---|---|
| Result | Factor | Measure |
| Accelerated Aging Factor, Q10\hat{ }factor | 7.0 | N/A |
| Accelerated Aging Time, AA in days | 104.8 | day |
| Accelerated Aging Time, AA in weeks | 15.0 | week |

Column functional tests were conducted on a weekly basis for up to 4 weeks and then every other week until week 15, as shown in the sampling schedule plan in Table 6.

Real time aging is done under ambient storage conditions (with nominal of 23° C.+9° C./−8° C.) for two years, plus one additional year, for a total of three years. The samples are drawn and assessed every three months for three years. See Table 6 for accelerated aging representation of real time aging with different storage condition, using the 010 rule and factor 4 (Table 5).

TABLE 6
Accelerated aging real time storage representation

| Aging Group | Aging Time | | Aging Time | | Aging Time | |
|---|---|---|---|---|---|---|
| Accelerated Aging (37° C.) | 4 | weeks | 8 | weeks | 15 | weeks |
| Real Time Aging Ambient | 6 | months | 12 | months | 24 | months |
| (23° C.) | | | | | | |

TABLE 7
Recovery of IgG (1 mg) using extraction in NBE format columns by Tecan
with 50 μL affinity chromatography sorbent comprising PMMA and recombinant
protein A after prolonged storage and accelerated aging. For every
time point, 12 measurements per sorbent lot have been collected. Shown
in the table are the means and standard deviations (STD).

| | Sorbent Lot 1 | | Sorbent Lot 2 | | Sorbent Lot 3 | | Combined | |
|---|---|---|---|---|---|---|---|---|
| | Mean | | Mean | | Mean | | Mean | |
| | Recovery | | Recovery | | Recovery | | Recovery | |
| Week | % | STD | % | STD | % | STD | % | STD |
| 0 | 83.6 | 0.04 | 82.3 | 0.11 | 81.1 | 0.05 | 82.3 | 0.07 |
| 1 | 86.4 | 0.07 | 83.2 | 0.08 | 83.3 | 0.05 | 84.3 | 0.07 |
| 2 | 81.7 | 0.06 | 77.2 | 0.15 | 78.5 | 0.08 | 79.1 | 0.10 |
| 3 | 84.1 | 0.06 | 81.6 | 0.07 | 79.5 | 0.07 | 81.7 | 0.06 |
| 4 | 83.0 | 0.14 | 85.3 | 0.06 | 85.4 | 0.11 | 84.6 | 0.11 |
| 6 | 80.1 | 0.27 | 79.9 | 0.06 | 79.5 | 0.05 | 79.8 | 0.16 |
| 8 | 84.4 | 0.06 | 81.5 | 0.08 | 81.9 | 0.05 | 82.6 | 0.06 |
| 10 | 80.6 | 0.11 | 80.5 | 0.08 | 78.5 | 0.12 | 79.9 | 0.11 |
| 12 | 82.6 | 0.07 | 84.2 | 0.05 | 83.1 | 0.07 | 83.3 | 0.06 |
| 14 | 78.9 | 0.05 | 80.3 | 0.04 | 80.1 | 0.03 | 79.8 | 0.04 |
| 15 | 81.4 | 0.25 | 80.1 | 0.26 | 81.5 | 0.04 | 81.0 | 0.21 |
| 17 | 83.5 | 0.04 | 83.9 | 0.04 | 82.8 | 0.04 | 83.4 | 0.04 |
| 19 | 82.1 | 0.05 | 81.0 | 0.03 | 82.0 | 0.05 | 81.7 | 0.04 |
| 21 | 82.2 | 0.05 | 83.0 | 0.05 | 82.4 | 0.04 | 82.5 | 0.05 |
| 23 | 81.4 | 0.011 | 81.0 | 0.09 | 82.2 | 0.03 | 81.5 | 0.08 |
| Mean | 82.4 | | 81.7 | | 81.5 | | 81.8 | |
| STD | | 0.12 | | 0.10 | | 0.06 | | 0.10 |
| Max | 86.4 | | 85.3 | | 85.4 | | 84.6 | |
| Min | 78.9 | | 77.2 | | 78.5 | | 79.1 | |

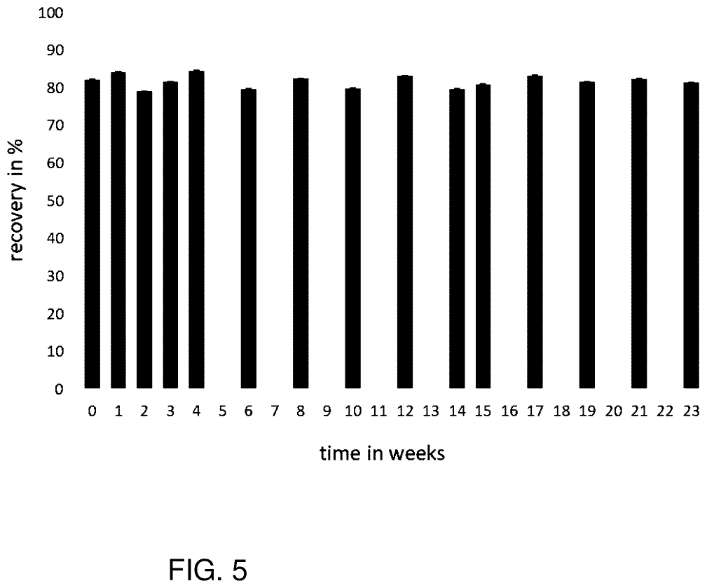

FIG. 5 shows the extraction recovery of IgG in % of 1 mg after prolonged periods of time of dry storing of affinity chromatography sorbent comprising PMMA and recombinant protein A in a narrow bore extraction (NBE) format column. Combined means from three affinity chromatography sorbent lots and error bars indicating combined standard deviations.

These results as shown in Table 7 and FIG. 5 demonstrate that the functional stability of NBE format columns by Tecan with 50 μL affinity chromatography sorbent comprising PMMA and recombinant protein A is maintained over the 23 weeks of storage under elevated temperatures. All three lots of columns show comparable performance in providing consistent IgG extraction recovery of 80% with little deviation, while no obvious functional deterioration is observed. The overall results therefore qualify all three lots of columns for a shelf life of at least three years. These results also displayed the robustness of the performance of the sample preparation workflow in using these Protein A columns.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### REFERENCE SIGNS

1. Reaction chamber
2. Filter
3. Narrow bore outlet
4. Air lock
5. Microparticulate sorbent

What is claimed is:

1. An affinity chromatography device for capturing biological molecules comprising
a carrier, and
a sorbent bed comprising
a solid support material; and
a ligand configured to bind specific biological molecules coupled to the support material,
wherein the sorbent bed is immobilized on or in the carrier, and
wherein the sorbent bed is in a dry format.

2. The affinity chromatography device of claim 1, wherein the ligand coupled to the solid support material comprises nickel or nickel-nitrilotriacetic acid (Ni-NTA), glutathione, streptavidin, a protein or antibody or a fragment thereof.

3. The affinity chromatography device of claim 1, wherein the ligand coupled to the solid support material is an immunoglobulin-binding protein.

4. The affinity chromatography device of claim 3, wherein the immunoglobulin-binding protein can bind an Fc region of an immunoglobulin.

5. The affinity chromatography device of claim 3, wherein the immunoglobulin-binding protein comprises protein A, protein G, protein L, or a truncated protein A, protein G, or protein L.

6. The affinity chromatography device of claim 3, wherein the immunoglobulin-binding protein is protein A.

7. The affinity chromatography device of claim 1, wherein the ligand coupled to the solid support material is covalently or non-covalently linked and immobilized thereon.

8. The affinity chromatography device of claim 1, wherein the solid support material comprises a polymer.

9. The affinity chromatography device of claim 8, wherein the polymer is selected from polymethyl methacrylate (PMMA), styrene-divinylbenzene copolymers (PS-DVB), hydroxylated DVB-PS, pyrrolidone-DVB based copolymers, polystyrenesulfonate (PSS), polythiophene dioxin (poly-2,3-dihydrothieno(3,4-b)-dioxin), poly-styrene-co-methyl methacrylate (PS-co-PMMA), polyaniline (PANI), poly(acrylic acid) (PAA), polyethylene (PE), polyvinyl chloride (PVC), polyethylene glycol (PEG), polythiophenes, and polyvinyl alcohol (PVA).

10. The affinity chromatography device of claim 8, wherein the polymer is polymethyl methacrylate (PMMA).

11. The affinity chromatography device of claim 1, wherein the solid support material comprises rigid beads.

12. The affinity chromatography device of claim 1, wherein the carrier is selected from a plate, a tube, a column, a well, a pipette tip, and a multi-well plate.

13. The affinity chromatography device of claim 1, wherein the carrier comprises polystyrene or glass.

14. The affinity chromatography device of claim 1, wherein the biological molecules are proteins.

15. The affinity chromatography device of claim 1, wherein the biological molecules are immunoglobulins.

16. A method for producing an affinity chromatography device for capturing biological molecules,
the method comprising:
coupling a ligand to a solid support material to form a sorbent bed in an aqueous medium, wherein the ligand is configured to bind specific biological molecules;
immobilizing the sorbent bed on or in a carrier; and
drying the sorbent bed.

17. A method for producing an affinity chromatography device for capturing biological molecules,
the method comprising:
(a) coupling a ligand to a solid support material to form a sorbent bed in an aqueous medium, wherein the ligand is configured to bind specific biological molecules;
(b) drying the sorbent bed to produce a dry powder;
(c) reconstituting the dry powder in an aqueous medium;
(d) immobilizing the sorbent bed on or in a carrier; and
(e) drying the sorbent bed.

18. The method for producing an affinity chromatography device of claim 16, wherein the drying the sorbent is in ambient conditions.

19. A method for determining the presence of a specific biological molecule in a sample, the method comprising:
(a) reconstituting an affinity chromatography device for capturing biological molecules in an aqueous medium, wherein the device comprises:
a carrier, and
a sorbent bed comprising
a solid support material; and
a ligand configured to bind specific biological molecules coupled to the support material,
wherein the sorbent bed is immobilized on or in the carrier, and
wherein the sorbent bed is in a dry format;
(b) contacting the sorbent bed with the sample, wherein the ligand binds specifically to the specific biological molecule when it is present; and
(c) determining if the ligand bound the specific biological molecule, wherein if the ligand bound the molecule, the specific biological molecule is determined to be present in the sample.

20. An automated laboratory system for detecting the presence of a specific biological molecule in a sample comprising the affinity chromatography device according to claim 1, wherein the sorbent bed is reconstituted in an aqueous medium.
